(19) United States
(12) Plant Patent Application Publication
(10) Pub. No.: US 2008/0141426 P1
(43) Pub. Date: Jun. 12, 2008

(54) Sedum kamtschaticum fischer "TottoriFujita No1 and TottoriFujita No2"

(76) Inventor: Aiko Fujita, Tottori (JP)

Correspondence Address: Aiko Fujita, 574, Iwatsune Iwami-cho Iwami-gun, Tottori, 681-0052, JP

(21) Appl. No.: 12/002,051

(22) Filed: Dec. 17, 2007

(30) Foreign Application Priority Data
Jun. 23, 2005 (JP) 16646
Jun. 23, 2005 (JP) 16667

(51) Int. Cl. A01H 5/00

(52) U.S. Cl. PLT/479

(57) ABSTRACT

New and distinct varieties of Sedum Kamtschaticum Fischer plant called “TottoriFujita No1 and TottoriFujita No2” are described. The two varieties which have the same properties except the size of their shoots diameter are characterized by their evergreen property, and having narrower internodes and more dwarf than their parent plant.

### CROSS REFERENCE TO RELATED APPLICATIONS

| | | | |
|---|---|---|---|
| 4,135,330 | January 1979 | Aoyama | 47/1 R |
| PTC/US2001/022800 | July 2001 | Mischo | A01G 1/100 |
| PP17,315 P2 | December 2006 | Oudshoorn | Plt/356 |

### STATEMENT REGARDING FEDERALLY-SPONSORED RESEARCH AND DEVELOPMENT

None

### LATIN NAME OF THE GENUS AND SPECIES OF THE PLANT CLAIMED

Latin name of the genus: Sedum

Specie of the plant: Kamtschaticum Fischer

Family: Crassulaceae

### LATIN NAME OF THE GENUS AND SPECIES OF THE PLANT CLAIMED

Name of the varieties to be registered: TottoriFujita No1 and TottoriFujita No2

### BACKGROUND OF THE INVENTION

1. Field of the Invention.

The use of vegetation as a bio-engineering is becoming increasingly popular in many developed countries. On the other hand, roof-garden laid on the roof of building is becoming increasingly popular in these countries. The interest for vegetation cover has been related to their capacity to reduce runoff volumes and peak flows, mitigate urban heat island effects and cool buildings during summer months. Vegetation cover can also be designed to improve urban biodiversity.

Sedum Kamtschaticum Fischer has been attracted as suitable plant for green roofs and embankments cover in Japan since 2000 to improve the country environment due to their characteristics such as drought tolerant, surviving under harsh conditions, growing in poor and stony soil and more importantly their evergreen property.

2. Description of Relevant Prior Art Including Information Disclosed Under 37 CFR 1.97 and 1.99.

Sedum Kamtschaticum Fischer can be reproduced either by sexually and/or asexually. One of parent plants of the claimed plants, which are excluded from the patent, is wildly growing in the areas of Niigata and Toyama prefectures in Japan. While the father plant is a wild originated from Tottori prefecture, however, it is important to note that its characteristic is not known yet. This parent plant is also known as orange stonecrop due to their orangey-yellow flower in summer. They are widely used in green roofs due to its drought tolerance and they can survive also in the harsh conditions such as a very cold to hot temperature, however they do not have the evergreen property.

In general, Sedum Kamtschaticum Fischer has a feature in which the sucker grows from winter while at the same season their leave is fallen. However, it was found after plant breeding that the offspring exhibits an evergreen characteristic. The winter sucker of the offspring starts growing in autumn and keeps greening until the coming winter and then fully grows at the early spring. In general, the leaves of this new plant are fallen in winter while at the same time their sucker is developed. On the other hand, this offspring has narrower internodes and has also more dwarf than their parent from Niigata and Toyama. The characteristics of the plant are shown in the data sheet “Characteristics tables 1 and 2”.

2.1 Cultivation Process of the Plant Breeding

2.1.1 Breeding Parental

Mother: Wild Sedum Kamtschaticum Fischers plant naturally grows in Kashiwasaki-city, Niigata prefecture, Japan

Father: Unknown

Genealogy of Breeding

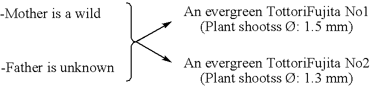

2.1.2 Process of the Mother Plant Seedling

The seedling of the mother plant was taken from Kashiwazaki City of Niigata Prefecture in July 1994. It is important to mention that this mother plant grew in this field with natural crossing multiplication. One hundred and twenty seeds of this plant were sown in November, 1996 at Iwatsune Iwami-chou Iwami-gun City of Tottori Prefecture.

2.1.3 Condition of Cultivation to Test the Characteristics of the TottoriFujita No1 and No2

Place of Cultivation: 360, Iwatsune Iwami-chou Iwami-gun, Tottori 681-0052, Japan

Period of Cultivation: April 1998 - March 2003

Method of Cultivation:

The mother plants were seeded into pots in Nov 1996 while the evergreens TottoriFujita No1 and TottoriFujita No2 were planted in April 1998. It was found through the test during April 1998 to March 2003 that both evergreens exhibit narrower internodes and more dwarf than the original one. On the other hand, it was noticed that TottoriFujita No1 and TottoriFujita No2 could be classified clearly by their difference in diameter of the plant shoots. TottoriFujita No1 exhibits a large shoots diameter (Ø:1.5 mm) compared to TottoriFujita No2 (Ø: 1.3 mm).

Breeding Method:

The breeding method was proceeded either by seed breeding (the seed is obtained by intercrossing a few plant varieties) or by nutrient (the seedling is from a seed of fungus)

Note: Maintenance and preservation conditions of the neither plant body used to verify the characteristics of the plant variety in the neither case whose seedlings are not seed nor fungus:

The Place of the maintenance and preservation methods is in the green house located in 360, Iwatsune Iwami-chou Iwami-gun, Tottori, Japan.

The parent plant is maintained in the green house condition.

### SUMMARY OF THE INVENTION

Two different Sedum Kamtschaticum Fischers, which is named TottoriFujita No1 and TottoriFujita No2, were obtained after breeding a wild mother Sedum Kamtschaticum Fischers with an unknown father Sedum. It was found and confirmed that both offspring exhibit an evergreen characteristic since their parents do not. This offspring has narrower internodes and more dwarf than their parent plant. On the other hand, it was noticed that TottoriFujita No1 and TottoriFujita No2 could be clearly classified by their difference in diameter of the plant shout. TottoriFujita No1 exhibits a large shoots diameter (Ø: 1.5 mm) as compared to TottoriFujita No2 (527 : 1.3 mm).

Asexual reproduction of the new varieties by terminal cutting at Tottori, Japan, since 2003, has shown that the unique features of these offspring are stable and reproduce true to type in successive generations.

Sedum Kamtschaticum Fischers has been considered as a suitable plant cover for green roofs and embankments cover. Indeed, the new plant varieties TottoriFujita No1 and TottoriFujita No2 give more promising outcome in the international greening business as a result of their particular characteristics as compared to their parent. Furthermore, the difference in shoots diameter between the two plant breeds provides a clear classification about their utility for the greening purpose. As the TottoriFujita No1 has the large diameter (Ø: 1.5 mm) than the TottoriFujita No2 (Ø: 1.3 mm), it is recommended to use for embankment cover while the last plant variety is generally for green roofs.

### BRIEF DESCRIPTION OF THE DRAWING

Some drawing descriptions showing the difference in characteristics between the TottoriFujita No1 and TottoriFujita No2 with their mother are highlighted in the following photographs.

FIG. 1 is a photograph showing the shoots morphology such as shoots diameter and leaf arrangement of the mother (opposite-decussate), TottoriFujita No1 (alternate) and TottoriFujita No2 (alternate).

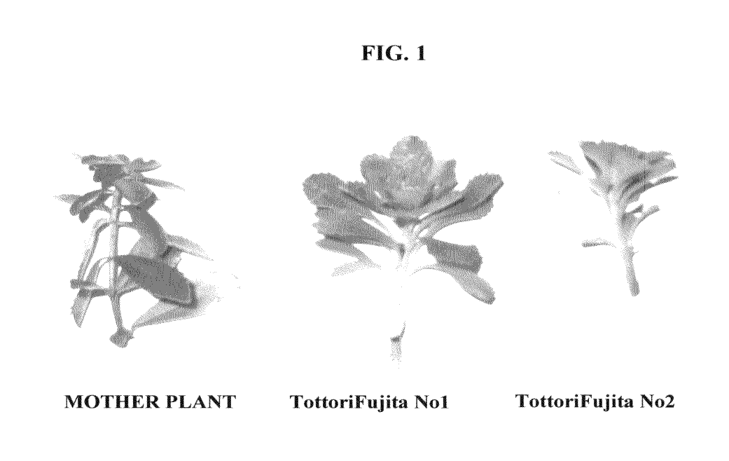

FIG. 2 is a photograph highlighting the characteristics of the face side of leaf such as margin and petiole of the mother, TottoriFujita No2 and TottoriFujita No1.

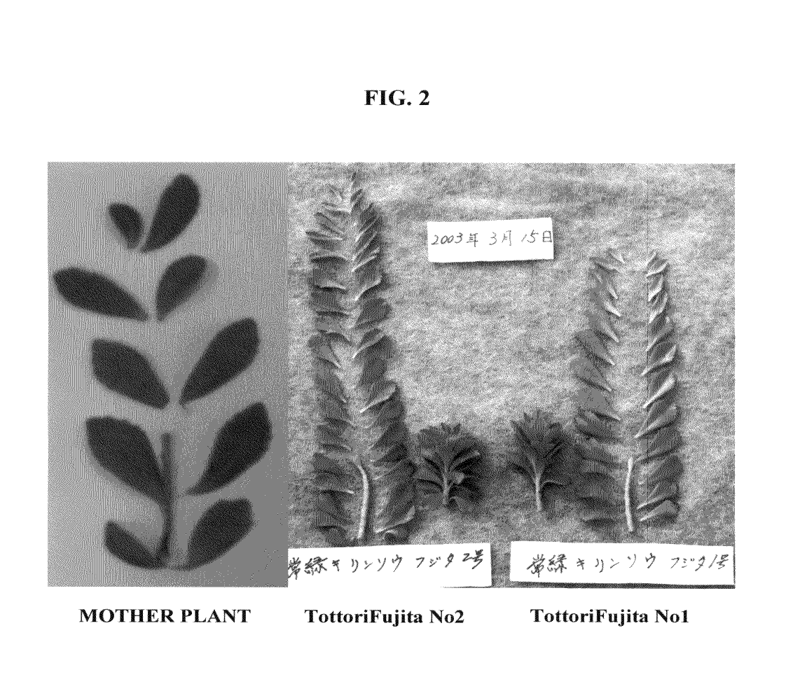

FIG. 3 is a photograph highlighting the characteristics of the opposite side of leaf such as midrib and blade of the mother, TottoriFujita No1 and TottoriFujita No2.

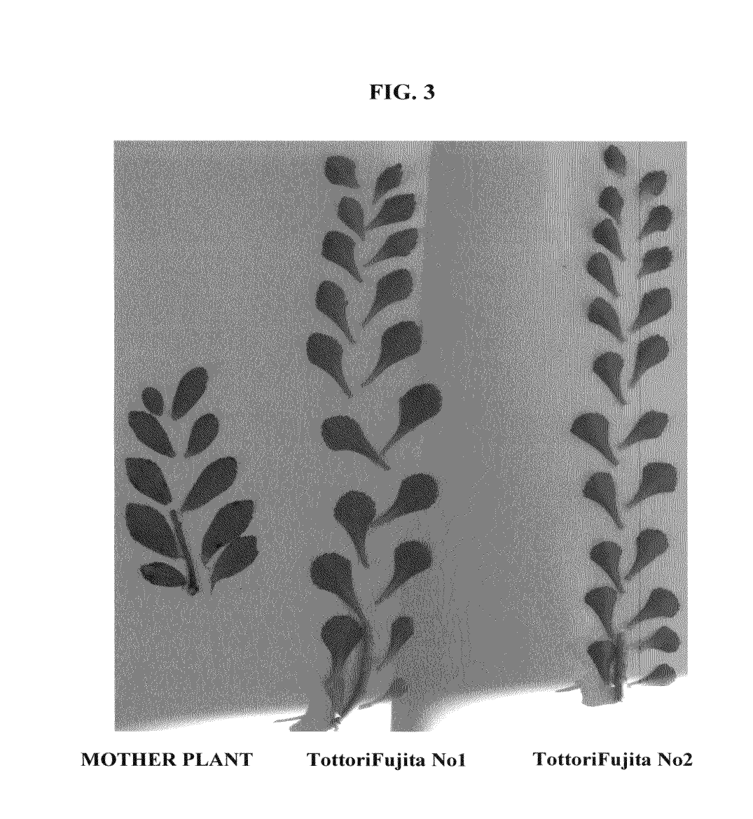

FIG. 4 is a photograph highlighting the difference in plant growth during winter between TottoriFujita No1 and the mother plant.

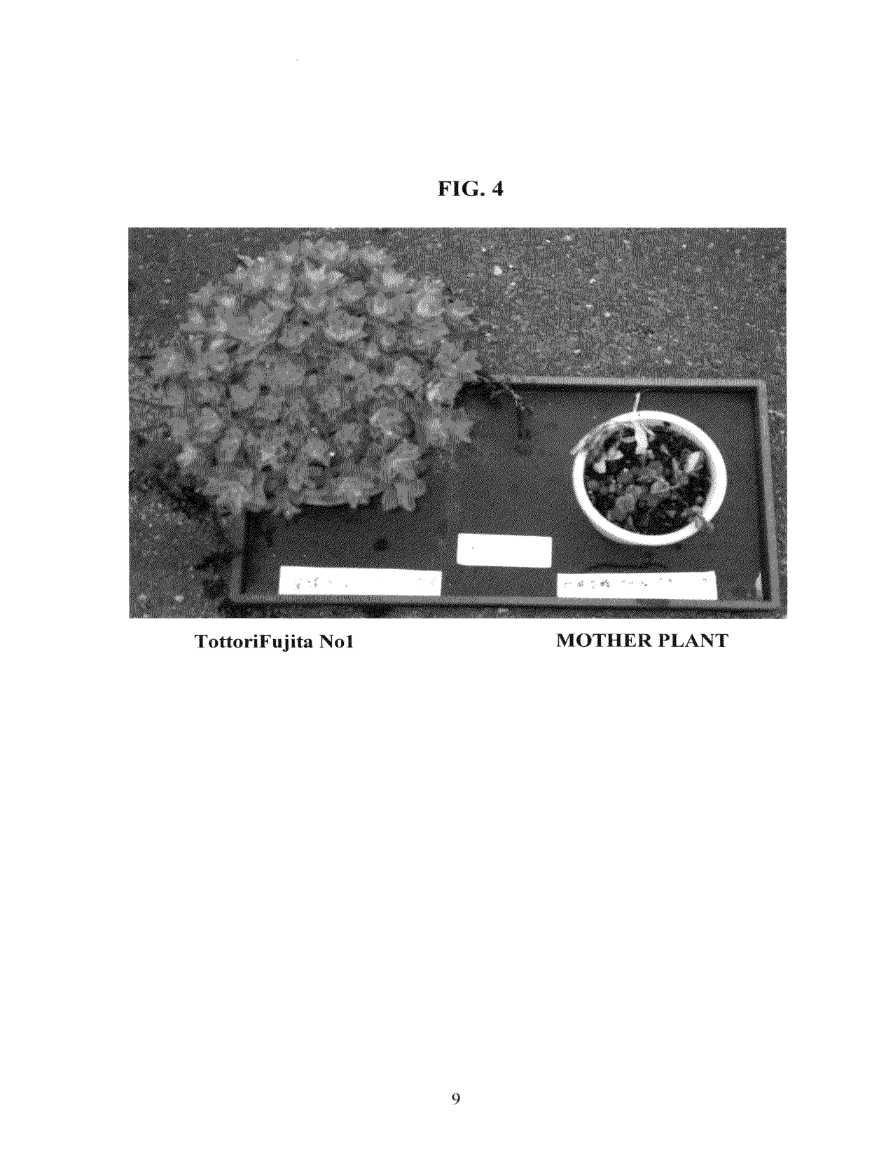

FIG. 5 is a photograph highlighting the difference in plant growth during winter between TottoriFujita No2 and the mother plant.

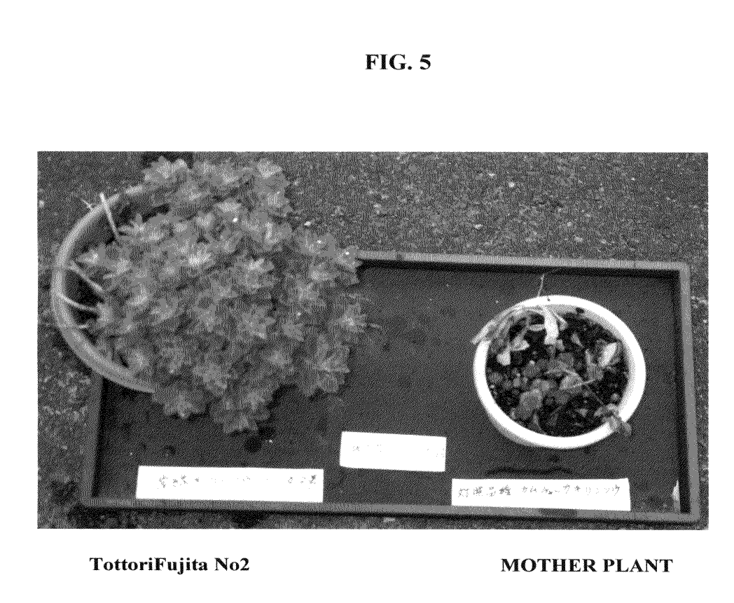

FIG. 6 is a photograph showing the difference in plant growth such as leaf size and plant shoots height between the two offspring; TottoriFujita No2 and TottoriFujita No1.

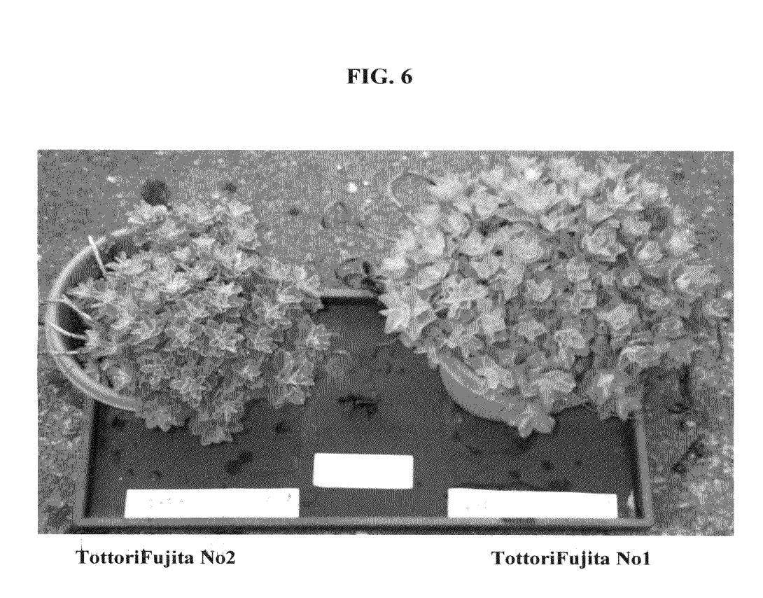

### DETAILED BOTANICAL DESCRIPTION OF THE PLANT

Sedum Kamtschaticum Fischer has a feature in which the sucker grows from winter while at the same season their leave is fallen. More importantly, it was found that the present offspring exhibits an evergreen characteristic. This new characteristic is highlighted with the winter sucker that starts growing in autumn and keeps greening until the coming winter and then fully grows at the early spring. In general, the leaves of this plant are fallen in winter while at the same time their sucker is developed. On the other hand, this offspring has narrower internodes and has also more dwarf than their parent from Niigata and Toyama. The botanical descriptions of the new plant variety characteristics are shown with their mother plant in the data sheet “Characteristics table 1” in the case of “TottoriFujita No1” and “Characteristics table 2” in the case of “TottoriFujita No2”.

According to the present characteristics such as drought tolerance and they can survive also in the harsh conditions such as a very cold to hot temperature, and evergreen property, these plant varieties are highly suitable for the international greening business. Furthermore, the deference in diameter between the two offspring classifies their use; TottoriFujita No1 is recommended for embankment cover while TottoriFujita No2 is generally recommended for green roofs.

CHARACTERISTIC TABLE 1
Name of plant specie: Sedum Kamtschaticum Fischer
Name of application kind: TottoriFujita No1
Applicant name: Aiko Fujita
Cultivator: Michiaki Fujita
Address of the applicant: 574, Iwatsune Iwami-chou Iwami-gun, Tottori, Japan
Place of cultivation: 360, Iwatsune Iwami-chou Iwami-gun, Tottori, Japan
Place of plant characteristic investigation:
360, Iwatsune Iwami-chou Iwami-gun, Tottori, Japan
Name of investigator: Michiaki Fujita
Date of the investigation: April, 1998

| | An attribute of application plant | | | | | | | | | Remark | An attribute of contrast plant | |
|---|---|---|---|---|---|---|---|---|---|---|---|---|
| Vegetative characteristic | 01 | 02 | 03 | 04 | 05 | 06 | 07 | 08 | 09 | Measured value | (Mother from Niigata) | (Mother from Toyama) |
| Shoot morphology | | | ascending | | Decumbent | | Displacement | | | 03 | 05 | 03 |
| | | | | | | | downward | | | | | |
| leaf part length | | | Low | | Middle | | High | | | 21 cm | 19 cm | 25 cm |
| Shoot length | | | Low | | Middle | | High | | | 28 cm | 26 cm | 32 cm |
| Width of turnip | | | Narrow | | Middle | | Wide | | | 39 cm | 35 cm | 47 cm |
| Degree of offshoot | | | Low | | Middle | | high | | | 05 | 05 | 05 |
| Generation of adventitious bud | None | Green leaf | Anthotaxis | | | | | | Other | 01 | 01 | 01 |
| Bristle on stem | None | | | | | | | | Having | 01 | 01 | 01 |
| Lignifications of stem | None | | | | | | | | Having | 09 | 09 | 09 |
| Leaf shape | Linear | oblong | Elliptic | Ovate | Orbicular | Spathulate | Palmate | | | 06 | 06 | 06 |
| Blade length with or without | None | | Short | | Middle | | Long | | | 6 mm | 6 mm | 6 mm |
| presence of petiole | | | | | | | | | | | | |
| Lamina length | Extremely | | Short | | Middle | | Long | | Extremely | 3.8 cm | 3.9 cm | 5 cm |
| | short | | | | | | | | Long | | | |
| Width of leaf | Extremely | | Narrow | | Middle | | Wide | | Extremely | 1.4 cm | 1.9 cm | 2.2 cm |
| | narrow | | | | | | | | wide | | | |
| Color on leaf surface | | | Pale-green | | Green | | Dark green | | Other | JHS Color | JHS Color | JHS Color |
| | | | | | | | | | | chartNO3712 | chartNO3712 | chartNO3711 |
| | | | | | | | | | | GreenYellow | GreenYellow | Deep yellowgreen |
| Red color developing on leaf | None | Temporarily | Whole year | | | | | | | 02 | 02 | 02 |
| surface | | | | | | | | | | | | |
| Color on back leaf side | | | Pale-green | | Green | | Dark green | | Other | JHS Color | JHS Color | JHS Color |
| | | | | | | | | | | chartNO3709 | chartNO3513 | chartNO, 3512 |
| | | | | | | | | | | LightGreen | Almond green | Verdant green |
| Red color developing on back | None | Temporarily | Whole year | | | | | | | 01 | 01 | 01 |
| side of leaf | | | | | | | | | | | | |
| Bristle-tipped leaves | None | | | | | | | | Having | 01 | 01 | 01 |
| White powdery substance on | None | | | | | | | | Having | 01 | 01 | 01 |
| leaf (pruinose) | | | | | | | | | | | | |
| Direction of the lateral warping | None | Introrse | Extrorse | | | | | | | 02 | 02 | 02 |
| of the leaves | | | | | | | | | | | | |
| Contortion of the plant leaf (leaf | None | | | | | | | | Having | 01 | 01 | 01 |
| epinasty) | | | | | | | | | | | | |
| Thickness of leaf | | | Thin | | Middle | | Thick | | | 1.2 mm | 1 mm | 0.9 mm |
| Number of serrate on leaf | None | | | | | | | | Having | 09 | 09 | 09 |
| margin | | | | | | | | | | | | |
| Shape of serrate on leaf margin | Serrate | Double | Dentate | Crenate | | | | | | 03 | 03 | 02 |
| | | serrate | | | | | | | | | | |
| Depth of serrate on leaf margin | | | Shallow | | Middle | | Deep | | | 05 | 05 | 05 |
| Number of serrate on leaf | | | Few | | Middle | | Many | | | 05 | 05 | 05 |
| margin | | | | | | | | | | | | |
| Shape of leaf apex | Sharpe | Obtuse | Orbicular | | | | | | | 03 | 03 | 03 |
| Shape of leaf base | Wedged | Obtuse | Orbicular | Truncate | Peltate | | | | | 01 | 01 | 02 |
| Speckle leaf | None | | | | | | | | Having | 01 | 01 | 01 |
| Length of the anthotaxy | | | Short | | Middle | | Long | | | 4.9 cm | 3.8 cm | 5.1 cm |
| (Inflorescence) | | | | | | | | | | | | |
| Number of flower | | | Few | | Middle | | Many | | | 117 | 117 | 117 |
| Maximum width of anthotaxy | | | Narrow | | Middle | | Wide | | | 9 cm | 6.3 cm | 9.4 cm |
| Minimum width of anthotaxy | | | Narrow | | Middle | | Wide | | | 6 cm | 5.2 cm | 7.1 cm |
| Color of rachis | Pale-green | Green | Green dark | Ecru | Pale red | Auburn | | | Other | JHS color | JHS color | JHS color |
| | | | blue | | | | | | | chartsNO3710 | chartsNO3710 | chartsNO3710 |
| | | | | | | | | | | YellowGreen | YellowGreen | YellowGreen |
| White powder on rachis | None | | | | | | | | Having | 01 | 01 | 01 |
| Direction of flower | Upward | Downward | | | | | | | | 01 | 01 | 01 |
| Diameter of flower | | | Short | | Middle | | Long | | | 1.5 cm | 1.1 cm | 1.6 cm |
| Shape of flower | Cartridge | Campanulate | Jar type | | Stelliform | | | | | 05 | 05 | 05 |
| Length of floral tube | | | Short | | Middle | | Long | | | 03 | 03 | 03 |
| Color on the surface of floral | White | Yellow | Light | Red | Peach | Purple | | | Other | JHS Color | JHS Color | JHS Color |
| tube | | | | | | | | | | chartNO2205 | chartNO2205 | chartNO2205 |
| | | | | | | | | | | Brilliant | Brilliant luteofulvous | Brilliant luteofulvous |
| | | | | | | | | | | luteofulvous | | |
| Color inside of floral tube | White | Yellow | Light | Red | Peach | Purple | | | Other | JHS Color | JHS Color | JHS Color |
| | | | | | | | | | | chartNO2205 | chartNO2205 | chartNO2205 |
| | | | | | | | | | | Brilliant | Brilliant luteofulvous | Brilliant luteofulvous |
| | | | | | | | | | | luteofulvous | | |
| Number of corolla lobes | 4 | Mixing | 5 | | | | | | | 03 | 03 | 03 |
| Edge shape of the corolla lobe | Linear | Obtuse | Circular form | Slightly | cuspidate | Concave | | | | 01 | 01 | 01 |
| | | | | cuspidate | | and | | | | | | |
| | | | | | | Slightly | | | | | | |
| | | | | | | cuspidate | | | | | | |
| Length of corolla lobe | | | Short | | Middle | | Long | | | 05 | 05 | 05 |
| Width of corolla lobe | | | Narrow | | Middle | | Wide | | | 05 | 05 | 05 |
| Color inside of the corolla | White | Yellow | Light | Red | Peach | Purple | | | Other | JHS Color | JHS Color | JHS Color |
| lobe | | | | | | | | | | chartNO2205 | chartNO2205 | chartNO2205 |
| | | | | | | | | | | Brilliant | Brilliant luteofulvous | Brilliant luteofulvous |
| | | | | | | | | | | luteofulvous | | |
| Color on the surface of corolla | White | Yellow | Light | Red | Peach | Purple | | | Other | JHS Color | JHS Color | JHS Color |
| lobe | | | | | | | | | | chartNO2205 | chartNO2205 | chartNO2205 |
| | | | | | | | | | | Brilliant | Brilliant luteofulvous | Brilliant luteofulvous |
| | | | | | | | | | | luteofulvous | | |
| Warping of the corolla lobe | None | Slightly | Inversion | | | | | | | 01 | 01 | 01 |
| | | inversion | | | | | | | | | | |
| Number of Striae on the corolla | None | | | | | | | | Having | 01 | 01 | 01 |
| Thrum flowers of the corolla | None | | | | | | | | Having | 01 | 01 | 01 |
| Visibility of anther | None or | | | | | | | | Visible | 09 | 09 | 09 |
| | unremarkable | | | | | | | | | | | |
| Quantity of pollen | | | Small | | Middle | | Much | | | 05 | 05 | 05 |
| Shape of calyx (sepal) | Linearly | lanceolate | Elliptically | Ovate | Elliptic | | | | | 02 | 02 | 02 |
| | lanceolate | | lanceolate | | | | | | | | | |
| Opening characteristic of the | After | After | It opens at the | | | Stelliform | | | | 06 | 06 | 06 |
| calyx | flowering, | flowering, it | bud | | | | | | | | | |
| | it doesn't | opens | | | | | | | | | | |
| | open | | | | | | | | | | | |
| Length of calyx | | | Short | | Middle | | Long | | | 05 | 05 | 05 |
| Color of calyx | Pale-green | Green | Iris | | | | | | Other | JHS Color | JHS Color | JHS Color |
| | | | | | | | | | | chartNO4305 | chartNO4305 | chartNO4305 |
| | | | | | | | | | | Brilliant green | Brilliant green | Brilliant green |
| Red thrum setting on calyx | No | Only on the | Only on the | Only on | Extensively | | | | | 01 | 01 | 01 |
| | coloration | margin | base | the point | | | | | | | | |
| Thrum of calyx | None | | | | | | | | Having | 01 | 01 | 01 |
| Time of flowering | | | Early | | Middle | | Late | | | 07 | 07 | 07 |
| shot-day response | | | Short | | Middle | | Long | | | 07 | 07 | 07 |
| Heat resistance | | | Weak | | Middle | | Strong | | | 07 | 07 | 07 |
| Cold resistance | | | Weak | | Middle | | Strong | | | 07 | 07 | 07 |
| Moisture resistance | | | Weak | | Middle | | Strong | | | 07 | 07 | 07 |
| Disease-resistance | | | Weak | | Middle | | Strong | | | 07 | 07 | 07 |
| Insect resistance | | | Weak | | Middle | | Strong | | | 07 | 07 | 07 |

CHARACTERISTIC TABLE 2
Name of plant specie: Sedum Kamtschaticum Fischer
Name of application kind: TottoriFujita No2
Applicant name: Aiko Fujita
Cultivator: Michiaki Fujita
Address of the applicant: 574, Iwatsune Iwami-chou Iwami-gun, Tottori, Japan
Place of cultivation: 360, Iwatsune Iwami-chou Iwami-gun, Tottori, Japan
Place of plant characteristic investigation:
360, Iwatsune Iwami-chou Iwami-gun, Tottori, Japan
Name of investigator: Michiaki Fujita
Date of the investigation: April, 1998

| | An attribute of application plant | | | | | | | | | Remark | An attribute of contrast plant | |
|---|---|---|---|---|---|---|---|---|---|---|---|---|
| Vegetative characteristic | 01 | 02 | 03 | 04 | 05 | 06 | 07 | 08 | 09 | Measured value | (Mother from Niigata) | (Mother from Toyama) |
| Shoot morphology | | | ascending | | Decumbent | | Displacement | | | 03 | 05 | 03 |
| | | | | | | | downward | | | | | |
| leaf part length | | | Low | | Middle | | High | | | 21 cm | 19 cm | 25 cm |
| Shoot length | | | Low | | Middle | | High | | | 28 cm | 26 cm | 32 cm |
| Width of turnip | | | Narrow | | Middle | | Wide | | | 39 cm | 35 cm | 47 cm |
| Degree of offshoot | | | Low | | Middle | | high | | | 05 | 05 | 05 |
| Generation of adventitious bud | None | Green leaf | Anthotaxis | | | | | | Other | 01 | 01 | 01 |
| Bristle on stem | None | | | | | | | | Having | 01 | 01 | 01 |
| Lignifications of stem | None | | | | | | | | Having | 09 | 09 | 09 |
| Leaf shape | Linear | oblong | Elliptic | Ovate | Orbicular | Spathulate | Palmate | | | 06 | 06 | 06 |
| Blade length with or without | None | | Short | | Middle | | Long | | | 6 mm | 6 mm | 6 mm |
| presence of petiole | | | | | | | | | | | | |
| Lamina length | Extremely | | Short | | Middle | | Long | | Extremely | 3.8 cm | 3.9 cm | 5 cm |
| | short | | | | | | | | Long | | | |
| Width of leaf | Extremely | | Narrow | | Middle | | Wide | | Extremely | 1.4 cm | 1.9 cm | 2.2 cm |
| | narrow | | | | | | | | wide | | | |
| Color on leaf surface | | | Pale-green | | Green | | Dark green | | Other | JHS Color | JHS Color | JHS Color |
| | | | | | | | | | | chartNO3712 | chartNO3712 | chartNO3711 |
| | | | | | | | | | | GreenYellow | GreenYellow | Deep yellowgreen |
| Red color developing on leaf | None | Temporarily | Whole year | | | | | | | 02 | 02 | 02 |
| surface | | | | | | | | | | | | |
| Color on back leaf side | | | Pale-green | | Green | | Dark green | | Other | JHS Color | JHS Color | JHS Color |
| | | | | | | | | | | chartNO3709 | chartNO3513 | chartNO, 3512 |
| | | | | | | | | | | LightGreen | Almond green | Verdant green |
| Red color developing on back | None | Temporarily | Whole year | | | | | | | 01 | 01 | 01 |
| side of leaf | | | | | | | | | | | | |
| Bristle-tipped leaves | None | | | | | | | | Having | 01 | 01 | 01 |
| White powdery substance on | None | | | | | | | | Having | 01 | 01 | 01 |
| leaf (pruinose) | | | | | | | | | | | | |
| Direction of the lateral warping | None | Introrse | Extrorse | | | | | | | 02 | 02 | 02 |
| of the leaves | | | | | | | | | | | | |
| Contortion of the plant leaf (leaf | None | | | | | | | | Having | 01 | 01 | 01 |
| epinasty) | | | | | | | | | | | | |
| Thickness of leaf | | | Thin | | Middle | | Thick | | | 1.2 mm | 1 mm | 0.9 mm |
| Number of serrate on leaf | None | | | | | | | | Having | 09 | 09 | 09 |
| margin | | | | | | | | | | | | |
| Shape of serrate on leaf margin | Serrate | Double | Dentate | Crenate | | | | | | 03 | 03 | 02 |
| | | serrate | | | | | | | | | | |
| Depth of serrate on leaf margin | | | Shallow | | Middle | | Deep | | | 05 | 05 | 05 |
| Number of serrate on leaf | | | Few | | Middle | | Many | | | 05 | 05 | 05 |
| margin | | | | | | | | | | | | |
| Shape of leaf apex | Sharpe | Obtuse | Orbicular | | | | | | | 03 | 03 | 03 |
| Shape of leaf base | Wedged | Obtuse | Orbicular | Truncate | Peltate | | | | | 01 | 01 | 02 |
| Speckle leaf | None | | | | | | | | Having | 01 | 01 | 01 |
| Length of the anthotaxy | | | Short | | Middle | | Long | | | 4.9 cm | 3.8 cm | 5.1 cm |
| (Inflorescence) | | | | | | | | | | | | |
| Number of flower | | | Few | | Middle | | Many | | | 117 | 117 | 117 |
| Maximum width of anthotaxy | | | Narrow | | Middle | | Wide | | | 9 cm | 6.3 cm | 9.4 cm |
| Minimum width of anthotaxy | | | Narrow | | Middle | | Wide | | | 6 cm | 5.2 cm | 7.1 cm |
| Color of rachis | Pale-green | Green | Green dark | Ecru | Pale red | Auburn | | | Other | JHS color | JHS color | JHS color |
| | | | blue | | | | | | | chartsNO3710 | chartsNO3710 | chartsNO3710 |
| | | | | | | | | | | YellowGreen | YellowGreen | YellowGreen |
| White powder on rachis | None | | | | | | | | Having | 01 | 01 | 01 |
| Direction of flower | Upward | Downward | | | | | | | | 01 | 01 | 01 |
| Diameter of flower | | | Short | | Middle | | Long | | | 13 cm | 1.1 cm | 1.6 cm |
| Shape of flower | Cartridge | Campanulate | Jar type | | Stelliform | | | | | 05 | 05 | 05 |
| Length of floral tube | | | Short | | Middle | | Long | | | 03 | 03 | 03 |
| Color on the surface of floral | White | Yellow | Light | Red | Peach | Purple | | | Other | JHS Color | JHS Color | JHS Color |
| tube | | | | | | | | | | chartNO2205 | chartNO2205 | chartNO2205 |
| | | | | | | | | | | Brilliant | Brilliant luteofulvous | Brilliant luteofulvous |
| | | | | | | | | | | luteofulvous | | |
| Color inside of floral tube | White | Yellow | Light | Red | Peach | Purple | | | Other | JHS Color | JHS Color | JHS Color |
| | | | | | | | | | | chartNO2205 | chartNO2205 | chartNO2205 |
| | | | | | | | | | | Brilliant | Brilliant luteofulvous | Brilliant luteofulvous |
| | | | | | | | | | | luteofulvous | | |
| Number of corolla lobes | 4 | Mixing | 5 | | | | | | | 03 | 03 | 03 |
| Edge shape of the corolla lobe | Linear | Obtuse | Circular form | Slightly | cuspidate | Concave | | | | 01 | 01 | 01 |
| | | | | cuspidate | | and | | | | | | |
| | | | | | | Slightly | | | | | | |
| | | | | | | cuspidate | | | | | | |
| Length of corolla lobe | | | Short | | Middle | | Long | | | 05 | 05 | 05 |
| Width of corolla lobe | | | Narrow | | Middle | | Wide | | | 05 | 05 | 05 |
| Color inside of the corolla lobe | White | Yellow | Light | Red | Peach | Purple | | | Other | JHS Color | JHS Color | JHS Color |
| | | | | | | | | | | chartNO2205 | chartNO2205 | chartNO2205 |
| | | | | | | | | | | Brilliant | Brilliant luteofulvous | Brilliant luteofulvous |
| | | | | | | | | | | luteofulvous | | |
| Color on the surface of corolla | White | Yellow | Light | Red | Peach | Purple | | | Other | JHS Color | JHS Color | JHS Color |
| lobe | | | | | | | | | | chartNO2205 | chartNO2205 | chartNO2205 |
| | | | | | | | | | | Brilliant | Brilliant luteofulvous | Brilliant luteofulvous |
| | | | | | | | | | | luteofulvous | | |
| Warping of the corolla lobe | None | Slightly | Inversion | | | | | | | 01 | 01 | 01 |
| | | inversion | | | | | | | | | | |
| Number of Striae on the corolla | None | | | | | | | | Having | 01 | 01 | 01 |
| Thrum flowers of the corolla | None | | | | | | | | Having | 01 | 01 | 01 |
| Visibility of anther | None or | | | | | | | | Visible | 09 | 09 | 09 |
| | unremarkable | | | | | | | | | | | |
| Quantity of pollen | | | Small | | Middle | | Much | | | 05 | 05 | 05 |
| Shape of calyx (sepal) | Linearly | lanceolate | Elliptically | Ovate | Elliptic | | | | | 02 | 02 | 02 |
| | lanceolate | | lanceolate | | | | | | | | | |
| Opening characteristic of the | After | After | It opens at the | | | Stelliform | | | | 06 | 06 | 06 |
| calyx | flowering, | flowering, it | bud | | | | | | | | | |
| | it doesn't | opens | | | | | | | | | | |
| | open | | | | | | | | | | | |
| Length of calyx | | | Short | | Middle | | Long | | | 05 | 05 | 05 |
| Color of calyx | Pale-green | Green | Iris | | | | | | Other | JHS Color | JHS Color | JHS Color |
| | | | | | | | | | | chartNO4305 | chartNO4305 | chartNO4305 |
| | | | | | | | | | | Brilliant green | Brilliant green | Brilliant green |
| Red thrum setting on calyx | No | Only on the | Only on the | Only on | Extensively | | | | | 01 | 01 | 01 |
| | coloration | margin | base | the point | | | | | | | | |
| Thrum of calyx | None | | | | | | | | Having | 01 | 01 | 01 |
| Time of flowering | | | Early | | Middle | | Late | | | 07 | 07 | 07 |
| shot-day response | | | Short | | Middle | | Long | | | 07 | 07 | 07 |
| Heat resistance | | | Weak | | Middle | | Strong | | | 07 | 07 | 07 |
| Cold resistance | | | Weak | | Middle | | Strong | | | 07 | 07 | 07 |
| Moisture resistance | | | Weak | | Middle | | Strong | | | 07 | 07 | 07 |
| Disease-resistance | | | Weak | | Middle | | Strong | | | 07 | 07 | 07 |
| Insect resistance | | | Weak | | Middle | | Strong | | | 07 | 07 | 07 |

1. The new and distinct varieties of Sedum Kamtschaticum Fischer plant called “TottoriFujita No1 and TottoriFujita No2” as herein illustrated and described.
